(19) United States
(12) Patent Application Publication
(10) Pub. No.: US 2026/0328751 A1
(43) Pub. Date: Oct. 22, 2026

(54) NANOPORE STRUCTURE AND A METHOD FOR FABRICATING THE NANOPORE STRUCTURE

(71) Applicant: The Chinese University of Hong Kong, New Territories (CN)

(72) Inventors: Ho Pui Ho, Kowloon (CN); Jianxin Yang, New Territories (CN); Dehua Hu, Kowloon (CN); Tianle Pan, New Territories (CN); Wu Yuan, New Territories (CN)

(21) Appl. No.: 19/644,052

(22) Filed: Apr. 10, 2026

Related U.S. Application Data
(60) Provisional application No. 63/790,161, filed on Apr. 17, 2025.

(51) Int. Cl. G01N 33/487; B82Y 15/00; B82Y 40/00

(52) U.S. Cl. CPC G01N 33/48721; B82Y 15/00; B82Y 40/00

(57) ABSTRACT

A nanopore structure and a method for fabricating the nanopore structure. The nanopore structure includes: an aperture extending through a substrate, wherein the aperture defines a central axis; and a dwell zone defining the aperture, the dwell zone configured to increase a translocation time of an analyte passing through the aperture along the central axis.

### FIELD OF THE INVENTION

The present invention relates to a nanopore structure and a method for fabricating the nanopore structure. In particular, the present invention relates to a method for fabricating a nanopore structure having a dwell zone.

### BACKGROUND OF THE INVENTION

Solid-state nanopores are nanoscale apertures fabricated in thin membranes, typically silicon-based materials. They function as sensors by detecting changes in ionic current as individual molecules or analytes pass through the aperture under an applied electrical field. Fabricating nanopores with precise dimensions and specific structural features remains a challenge.

Conventional methods often lack control over the final pore size and geometry, potentially limiting their performance in applications such as single-molecule detection and sequencing. There is a need for improved fabrication techniques that allow for greater control over the nanopore structure, particularly features that influence the interaction time between the analyte and the sensor. The present invention addresses challenges in the field by providing a novel nanopore structure and a method for its fabrication, aiming to enhance sensing capabilities.

### SUMMARY OF THE INVENTION

In accordance with a first aspect, there is provided a nanopore structure comprising: an aperture extending through a substrate, wherein the aperture defines a central axis; and a dwell zone defining the aperture, the dwell zone configured to increase a translocation time of an analyte passing through the aperture along the central axis.

In accordance with the first aspect, the dwell zone corresponds to an elongated sensing length within the aperture and along the central axis.

In accordance with the first aspect, the elongated sensing length is greater than a narrowest opening diameter of the aperture.

In accordance with the first aspect, the aperture comprises a narrowest opening diameter of less than or equal to 4.4 nm.

In accordance with the first aspect, the elongated sensing length is in a range of 90-115 nm.

In accordance with the first aspect, the dwell zone is defined by a cylindrical tubular structure including the aperture extending therethrough along the central axis.

In accordance with the first aspect, the nanopore structure further comprises an entry zone arranged to guide the analyte towards an entrance of the dwell zone with the narrowest opening diameter of the aperture.

In accordance with the first aspect, the entry zone includes a funnel-shaped entry.

In accordance with the first aspect, the entry zone includes a micro-pyramidal shape formed by anisotropic etching of the substrate.

In accordance with the first aspect, the substrate includes a (100) silicon substrate.

In accordance with a second aspect, there is provided a method for fabricating a nanopore structure, comprising: forming an initial structure on a front-side of a (100) silicon substrate, wherein the initial structure includes a recess having a micro-pyramid shape; and etching a portion of substrate material from a back-side of the (100) silicon substrate to form the nanopore structure including an aperture extending through the (100) silicon substrate at a tip of the recess on the front-side.

In accordance with the second aspect, the nanopore structure further includes a dwell zone being form by etching the back-side of the (100) silicon substrate, wherein the aperture is extended along an elongated sensing length defined by the dwell zone, and wherein the dwell zone is configured to increase a translocation time of an analyte passing through the aperture.

In accordance with the second aspect, the step of etching a portion of substrate material from the back-side of the (100) silicon substrate comprises the step of performing a photovoltaic electrochemical etch-stop process to regulate an etching rate of the substrate material at or proximate to the aperture of the nanopore structure.

In accordance with the second aspect, the photovoltaic electrochemical etch-stop process includes, under an electrical bias Vp applied across the front-side and the back side of the (100) silicon substrate, an application of a near-field optical irradiation at or proximate to the aperture of the nanopore structure to induce a photoinhibition effect so as to locally modulate the etching rate at or proximate to the aperture of the nanopore structure.

In accordance with the second aspect, the electrical bias is a forward bias.

In accordance with the second aspect, the near-field optical irradiation includes narrowband blue light.

In accordance with the second aspect, the near-field optical irradiation is applied to the front-side of the (100) silicon substrate covering at least the tip of the recess to generation a near-field diffraction pattern around the tip, wherein a bright area is defined at or proximate to the tip.

In accordance with the second aspect, the near-field optical irradiation is applied to generate a photovoltage Vo induced by a separation of photogenerated electron-hole pairs which at least partially offsets the electrical bias Vp being applied to suppress the etching rate at the bright area.

In accordance with the second aspect, the near-field optical irradiation and the electrical bias induce a local photovoltage that suppresses the etching rate in illuminated areas (the bright areas) relative to non-illuminated areas (the dark areas).

In accordance with the second aspect, the method further comprises the step of thinning the (100) silicon substrate to a predetermined remaining thickness prior to the step of etching a portion of substrate material from the back-side of the (100) silicon substrate to form the nanopore structure.

In accordance with the second aspect, remaining thickness is detected by monitoring transmission spectral characteristics including a shift in transmission spectral peaks of broadband blue light detected by a spectrometer at the front-side of the (100) silicon substrate, wherein the broadband blue light is irradiated on the back-side of the (100) silicon substrate during the step of thinning the (100) silicon substrate.

In accordance with the second aspect, the (100) silicon substrate is etched by anisotropic wet etching using KOH.

In accordance with the second aspect, the predetermined remaining thickness is less than half a central wavelength of incident light used during the photovoltaic electrochemical etch-stop process.

In accordance with the second aspect, the predetermined remaining thickness is ranging from 19 nm to 114 nm.

In accordance with the second aspect, the method further comprising the step of depositing an etching-mask on the substrate before forming the initial structure.

In accordance with the second aspect, the etching-mask comprise silicon nitride.

In accordance with the second aspect, the method further comprises the step of performing over-etching and controlling an over-etching time after initial perforation of the aperture to fine-tune a final diameter of the nanopore aperture.

In accordance with the second aspect, controlling the over-etching time allows fabrication of nanopore apertures with diameters less than 10 nm.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the present invention will now be described, by way of example, with reference to the accompanying drawings in which:

FIG. 1A is a schematic illustration of the a nanopore structure, also known as MPSN, and a fabrication process for fabricating the nanopore structure, in which (i) illustrates the pre-etching process using KOH to achieve a remaining thickness H, which is the distance between the tip of the micro-pyramid and the silicon/etchant interface, ranging from 114 nm to 19 nm; (ii) and (iii) illustrate the local etching process using photoinhibition-assisted KOH etching to regulate the etching rate and final pore size, which is the narrowest opening diameter, of the nanopore structure, in accordance with embodiments of the present invention.

FIG. 1B presents representative transmission spectra used to closely monitor the remaining thickness during the pre-etching process, where a spectral peak λp of 520 nm corresponds to an H of 1270 nm.

FIG. 1C shows the measurement of the spectral peak using a spectrometer and the remaining silicon thickness using a contact profilometer, with black dots representing experimental data from 4 samples and the red line representing the fitting result. The standard deviation of the measured remaining silicon thickness at the tested spectral peak is approximately 8 nm based on four wafer tests.

FIG. 1D displays the current signal (Iₚ) measured during the local etching process, indicating that depending on the remaining thickness H, different irradiation times tᵢ are needed to achieve silicon perforation, leading to an over-etching stage signified by a current ramp with a slope tan θ_{I} over 0.5 nA/sec. The pore size of the MPSN is then precisely controlled by the over-etching time tₒ.

FIG. 1E is a front-view SEM image of a small MPSN S₀ with a pore size of 4.4 nm fabricated using a remaining thickness of 19 nm, an irradiation time of 20 s, and an over-etching time of 0.6 s.

FIG. 1F is a back-view TEM image of the small MPSN S₀.

FIG. 1G shows the pore size characterization using image grayscale analysis of the line in FIG. 1F.

FIG. 2A shows schematic back-view nanopore shapes (left) and corresponding TEM images (right) of MPSNs fabricated with different remaining thicknesses H (or irradiation times tᵢ) and over-etching times to.

FIG. 2B presents the side-view nanopore profile of MPSN S₁ derived from grayscale analysis, with an inset (black box) showing a schematic side-view nanopore profile.

FIG. 2C presents the side-view nanopore profile of MPSN S₂ derived from grayscale analysis, with an inset (black box) showing a schematic side-view nanopore profile.

FIG. 2D presents the side-view nanopore profile of MPSN S₃ derived from grayscale analysis, with an inset (black box) showing a schematic side-view nanopore profile.

FIG. 2E shows pore sizes versus combinations of H (or tᵢ) and tₒ, indicating three working regions for fabricating different pore sizes: sub-10 nm (i), between 10 to 40 nm (ii), and over-40 nm (iii).

FIG. 2F illustrates the increase in pore size with over-etching time to when H=19 nm (or tᵢ=20 s), with error bars representing the standard deviations of experimental data measured from 3 samples.

FIG. 3A is a plot showing the distribution of electric field in MPSN when the bias voltage is 0.3 V. The inset shows an enlarged view around the nanopore. The sensitive length L along the z direction is defined as the distance between the two points at which the electric field intensity is decayed to e^{4@} of the maximum intensity located in the nanopore centre.

FIG. 3B is an illustration of simulated translocation of a 7-nm circular molecule with surface charge of 17e through a location at z=85 nm by calculating spatial distribution of the net ionic concentration difference (v1c1+v2c2) in the MPSN within the area defined by x from −200 nm to 200 nm and z from −100 nm to 200 nm. Here, v1 and v2 represent the valences of potassium and chloride ions, respectively, while c1 and c2 represent the concentrations of potassium and chloride ions, respectively.

FIG. 3C is an illustration of simulated translocation of a 7-nm circular molecule with surface charge of 17e through a location at z=45 nm. Other conditions are the same as the simulation in FIG. 3B.

FIG. 3D is an illustration of simulated translocation of a 7-nm circular molecule with surface charge of 17e through a location at z=5 nm. Other conditions are the same as the simulation in FIG. 3B.

FIG. 3E is an illustration of simulated translocation of a 7-nm circular molecule with surface charge of 17e through a location at z=−15 nm. Other conditions are the same as the simulation in FIG. 3B.

FIG. 3F is a plot showing the corresponding current blockade change ΔI caused by the molecular translocation. The x- and z-direction are set to parallel to [0,1,0] and [1,0,0] crystal orientation of the silicon samples, respectively. The origin is positioned at the centre of the nanopore.

FIG. 4A illustrates a schematic of an experimental setting to study the light power dependence of photoinhibition-assisted KOH etching on silicon at room temperature. The remaining thickness H has known linear dependence on square side length a_{etch} measured in the silicon pyramid, i.e.,

$$\frac{2 H}{{a}_{etch}} = tan 54.7 ° ,$$

where the light power is 0 W.

FIG. 4B is illustrates a schematic of the experimental setting of FIG. 4A where the light power is 1 W.

FIG. 4C illustrates 16 microscopic photographs of etched silicon samples using different etching time and light power. The square sides are labelled with dashed white lines.

FIG. 4D is a plot illustrating the dependence of silicon thickness H on the etching time and light power. The fitted lines indicate that the etching rate decreases with the increase of light power. Light power of 0 W, 0.3 W, 0.6 W, and 0.9 W led to etching rates of 54 nm/min (black), 37 nm/min (red), 28 nm/min (blue), and 23 nm/min (purple), respectively.

FIG. 5A is an illustration showing time averaged Poynting vector <S> distribution when light with wavelength λ of 475 nm is incident from the back side boundary Z₂ to the front side boundary Z₁, thickness H of remaining silicon layer is 0.

FIG. 5B is a plot illustrating transmission spectrum in Z₁ for H=0, 100, 200, 300, and 400 nm when a broadband incident light with peak wavelength=460 nm and FWHM bandwidth=60 nm is incident from Z₂.

FIG. 5C illustrates <S> distribution when λ_{P}=475 nm light is incident from Z₁ at H=10 nm. Inset shows a ring-shape near-field diffraction pattern in the remaining layer at H=10 nm.

FIG. 5D is a plot showing the enlarged distribution curve of <S> along x from −65 nm to 65 nm in Z₂ of FIG. 5C. In the model, x=0 is set on the central axis.

FIG. 6A is a schematic diagram showing spectral a detection system for monitoring pre-etching process. The schematics of spectral detection system mainly consisting of broadband blue light source, optical alignment system, silicon sample, etchant, spectrometer, and CMOS.

FIG. 6B is a schematic diagram showing an electro-optical system for performing near-field photoinhibition-assisted electrochemical etching process. The schematics of electro-optical system mainly consisting of narrowband blue light source, photodiode, operational amplifier, and electrical circuit.

### DETAILED DESCRIPTION OF ILLUSTRATIVE EMBODIMENTS

The present invention relates to a nanopore structure and a method for its fabrication, particularly designed to enhance analyte detection by modulating translocation time and increasing capture radius.

The inventors, through their experiments and trials, devised that nanopore sensing is a technique that involves monitoring temporal traces of current blockades as molecules pass through a nanopore driven by forces acting on the molecules. A nanopore structure may be integrated into portable sensing devices with electronics which may be used in applications such as nanopore sequencing.

Nanopore devices may utilise electrokinetic force for single-molecule translocation to produce current blockade signals. However, it may be challenging to control the translocation speed, resulting in non-uniform conductance signals with limited temporal resolution and therefore low conformational sensitivity for discriminating molecular features. Although speed control with protein motors may be employed in biological nanopores, achieving a stable protein motor feed rate and high conductance drop as in solid-state nanopores remains challenging. On the other hand, controlled molecular translocation in nanopores may be employed using a nanopositioner or an atomic force microscope cantilever, however, tethered molecules are required, preventing complete translocation.

Without wishing to be bound by theory, a nanopore with the desired shape and size and made of an appropriate material is imperative for the accurate detection and characterisation of molecules in question, such as proteins. For example, on-film molecule sensing, a funnel-shaped nanopore may help enabling targets distributed over a large range to better address and pass through the nanopores.

In an alternative example, nanopores fabricated in ultrathin nanometre-thickness films, such as lipid bilayers, Si₃N₄, MoS₂ and graphene, offer desirable chemical stability against analytes, silicon nanopores fabricated in micrometre-thickness wafers in a reproducible and scalable manner provide better mechanical strength to withstand high levels of inertial force.

Anisotropic chemical etching is a technique for cost-effective fabrication of silicon nanopores. However, conventionally, this technique is suboptimal for fabricating silicon nanopores smaller than 8 nm owing to unavoidable over-etching. Alternatively, fabrication of truncated pyramidal nanopores with sub-5-nm dimensions on silicon-on-insulator substrates may be achieved using electron-beam lithography and less than 100-nm-thick membranes. However, the Debye length of truncated pyramidal nanopores is less than the nanopore size and the electroosmotic effect dominates the molecule translocation, resulting in short and unstable dwell times in readings of inadequate conformationally sensitive signals for discriminating molecules.

Preferably, methods such as elongating the sensing length of the nanopore, slowing the translocation speed by introducing new potential gradients, optimising test environments and fine-tuning molecular surface charges may be useful for extending the dwell times of molecules in nanopores, however, the stochastic nature of molecular dynamics in nanopores influences the translocation speed and limits high-fidelity and sensitive measurements of molecular conformation.

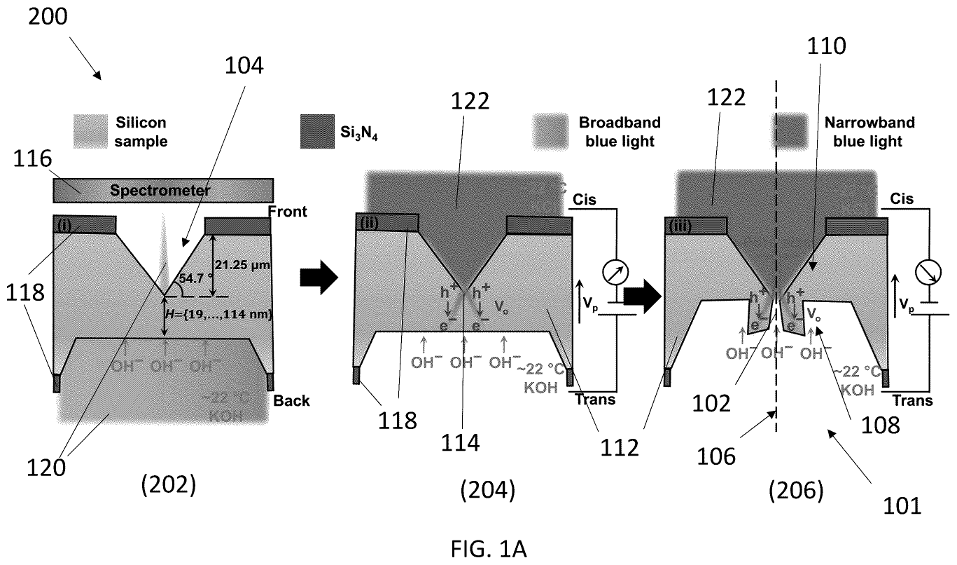

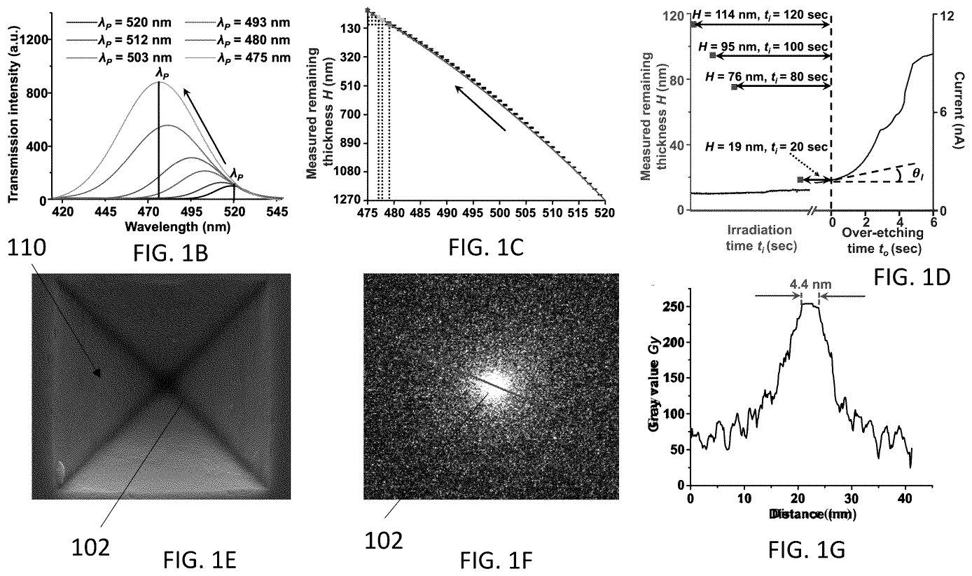

With reference to FIG. 1A, there is shown an example embodiment of a nanopore structure 101 being fabricated. The nanopore structure 101 comprises: an aperture 102 extending through a substrate 112, wherein the aperture 102 defines a central axis 106; and a dwell zone 108 defining the aperture 102, the dwell zone 108 configured to increase a translocation time of an analyte passing through the aperture 102 along the central axis 106.

It should be noted that the term “comprising” (and its grammatical variations) as used herein are used in the inclusive sense of “having” or “including” and not in the sense of “consisting only of”.

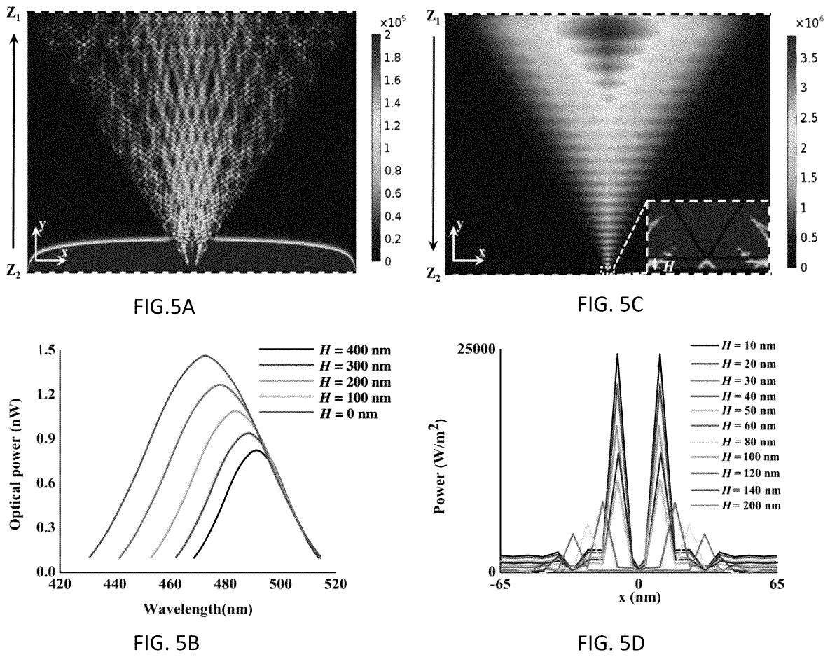

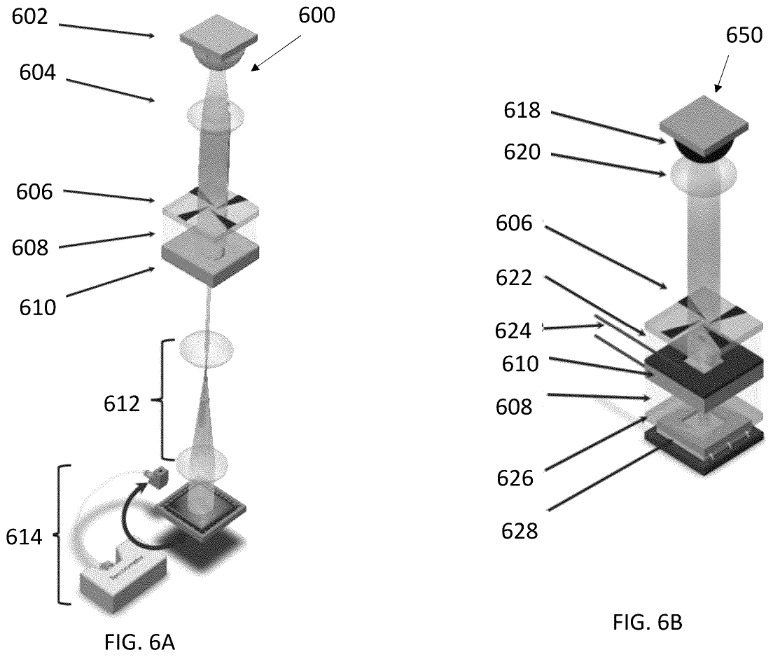

In this embodiment, the nanopore structure 101 comprises an aperture 102 extending through a substrate 112, defining a central axis, and a dwell zone 108 defining the aperture. This dwell zone 108 is specifically configured to increase the translocation time of an analyte passing through the aperture 102 along the central axis 106. This increased translocation time is advantageous for sensing applications, allowing for more detailed analysis of the analyte, as potentially illustrated in the sensing examples of FIG. 5 and FIG. 6.

With reference to FIG. 1A, the dwell zone 108 corresponds to an elongated sensing length within the aperture 102 and along the central axis 106. Preferably, this elongated sensing length is greater than the narrowest opening diameter of the aperture 102. This geometric configuration contributes directly to the increased dwell time of the analyte within the sensing region. For example, the nanopore may be defined with an aperture 102 with a diameter of around 5 nm, where the elongated sensing length or the dwell zone 108 may be of the length of around 100 nm.

With reference to FIGS. 1E to 1G, in an example nanopore structure 101 fabricated by the inventors, the nanopore structure 101 has an aperture 102 possesses a narrowest opening diameter a 4.4 nm pore size is shown via SEM (FIG. 1E), TEM (FIG. 1F) and greyscale analysis (FIG. 1G). Alternatively, the pore size or the narrowest opening diameter may be less than or equal to 10 nm, enabling the detection of single molecules.

Preferably, the elongated sensing length, may be preferably in the range of 90-115 nm. In one embodiment, the dwell zone 108 may be defined by a cylindrical tubular structure which includes the aperture 102 extending through it along the central axis 106. This tubular structure forms the elongated sensing region, where the molecules are allowed to travelled within the tubular structure like in a tunnel, therefore dwell time may be increased since the molecules in analyte have to travel along the entire length of the dwell zone 108 before exiting the nanopore.

Preferably, to facilitate the capture of analytes, the nanopore structure 101 may further comprise an entry zone 110, in connection with the dwell zone 108, at the narrowest opening diameter of the aperture. This entry zone 110 may guide the analyte towards the entrance of the dwell zone 108, specifically towards the narrowest opening diameter of the aperture 102. The entry zone 110 may include a funnel-shaped entry, e.g. a micro-pyramidal shape formed by etching.

In one example, the micro-pyramidal shape entry zone 110 may be fabricated by performing wet anisotropic etching of a (100) silicon substrate 112 using wet etchant containing potassium hydroxide (KOH), where KOH only selectively etch silicon along the (100) orientation but not the (111) orientation, resulting an angle of 54.7 degree from horizontal (100 orientation). The overall fabrication process resulting in this structure, including the entry zone and dwell zone, is schematically depicted in FIG. 1A.

The nanopore structure 101 may be fabricated by the process 200 in accordance with an example embodiment of the present invention, the method comprises the following steps of: forming an initial structure 104 on a front-side of a (100) silicon substrate 112, wherein the initial structure includes a recess having a micro-pyramid shape; and etching a portion of substrate 112 material from a back-side of the (100) silicon substrate 112 to form the nanopore structure 101 including an aperture 102 extending through the (100) silicon substrate 112 at a tip 114 of the recess on the front-side. In addition, the fabrication process 200 may also involve a pre-etching 202 and local etching steps 206, where thickness of various regions on the back-side of the silicon substrate 112 may be etched away in a controlled manner. The fabrication process generally involves the preparation process and pre-etching process followed by a local etching process, further described in details as follows.

Preferably, the etching from the back-side forms not only the aperture 102 but also the dwell zone, wherein the aperture 102 is extended along an elongated sensing length defined by the dwell zone. This configures the structure to increase analyte translocation time as described earlier.

In addition, before the main back-side etching step (or the local etching step), the method 200 may include thinning the (100) silicon substrate 112 to a predetermined remaining thickness, preferably between 19 nm and 114 nm, e.g. step 202 as shown in FIG. 1A. This thinning step may help controlling the final length of the dwell zone. The thinning, like other etching steps, can be performed using anisotropic wet etching, for example, with KOH as previously mentioned, and optionally, electrolyte such as KCl may be introduced in the etchant for altering the resultant ion concentration or electrical conductivity of the etchant.

Preferably, the remaining thickness can be monitored by detecting transmission spectral characteristics (e.g., peak shifts) of broadband blue light shone on the back-side and detected from the front-side during the thinning process, e.g. by using a spectrometer 116 in step 202. The predetermined remaining thickness influences the subsequent irradiation time (tᵢ) needed for perforation as illustrated in FIG. 1D.

For example, during the preparation process in an experiment performed by the inventors, two 200-nm thick silicon nitride (Si₃N₄) layers 118 were deposited on both sides of a (100) silicon wafer. Square patterns were then transferred onto the photoresist layers on silicon using photolithography. After removing the exposed Si₃N₄ layers through plasma etching, the silicon nitride etch mask 118 is formed with a squared opening, where an inverted micro-pyramid structure with a depth of approximately 21.25 μm was fabricated on the front side of silicon using 30% (w/w) KOH etchant heated to 80.0° C. Alternatively, the initial structure may be fabricated using other etching method such as dry which may etch away silicon in both vertical and horizonal directions. In this example, due to anisotropic wet etching effect, four pyramid side walls with an angle of 54.7 degree are formed, and the “base” of the pyramid is substantially the same as the squared opening of the silicon nitride etch mask.

In the pre-etching process, first 80.0° C. and then the room temperature (~22.0° C.) KOH etchant was used to etch silicon from the backside to achieve a remaining thickness of 19-114 nm. The length of the dwell zone 108 will also be determined by the remaining thickness before the local etching process that forms the dwell zone 108 or the tubular structure with the aperture 102 therethrough.

In this experiment, referring also to FIG. 1, a spectral detection system consisting of broadband blue light 120 and a spectrometer 116 were used to precisely track the transmission spectral characteristics of the remaining silicon, at step 202. The spectral peak was blue shifted during the etching process owing to the increased transmission of silicon for short light wavelengths as shown in FIG. 1B. With reference to FIG. 1C, it was observed that the spectral peak λₚ can be used to precisely measure the remaining thickness H because of their close correlation as follows:

$${λ}_{p} = - 5 4. {e}^{\frac{1270 - H}{2090.67}} + 574.$$

In one example, with reference to FIG. 1A, the fabrication method involves performing a photovoltaic electrochemical etch-stop process during the back-side etching step to regulate the etching rate at or proximate to the aperture. In this process, the etching rate may be controlled by applying an electrical bias (Vp) across the (100) silicon substrate 112 and using near-field optical irradiation near the forming aperture 102 to induce a photoinhibition effect, locally modulating the etching rate. The electrical bias is preferably a forward bias. Control signals related to this process, such as the measured current during local etching as illustrated in FIG. 1D, might be used to monitor the process.

Preferably, the near-field optical irradiation may be narrowband blue light 122 and applied to the front-side covering at least the tip 114 of the micro-pyramid recess, at step 204 before the local etching process and during the local etching process at step 206. Preferably, a near-field diffraction pattern may be generated around the tip 114, creating a bright area proximate to the tip 114. In this process, the irradiation generates a photovoltage (Vo) induced by separation of photogenerated electron-hole pairs, which at least partially offsets the applied electrical bias (Vp), thereby suppressing the etching rate specifically in the illuminated bright area. This precise, localized control allows for the formation of the desired elongated dwell zone 108 structure and control over the final pore size, enabling fine-tuning through controlling over-etching time after perforation is detected.

In the experiment performed, followed by the preparation step and pre-etching step as described earlier, a local etching process was subsequently performed using an electro-optical system comprising a narrowband blue light source for generating a near-field diffraction pattern on the silicon/etchant interface and an electrical circuit for applying a forward bias V_{P} of 800 mV and measuring the current signal Iₚ.

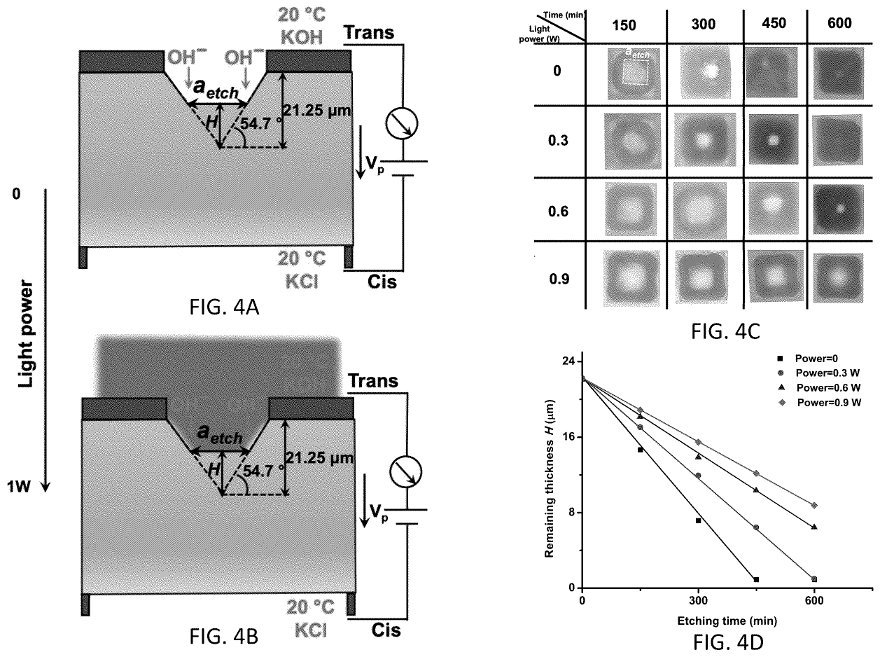

When the silicon sample is illuminated, the photo-voltage Vₒ induced by the separation of the photogenerated hole-electron pairs partially offsets the applied potential V_{P}, resulting in a slower etching rate (i.e., photoinhibition or photovoltaic electro-chemical etch-stop effect) on bright areas than on dark areas of the diffraction pattern around the pyramid tip 114. The photoinhibition effect assisted KOH etching rate is found inversely related to the optical power. In addition, the remaining thickness of silicon H determines the size of the central dark area on the silicon/etchant interface, which in turn defines the fast-etching area and eventually the pore size of nanopore structure 101. The photoinhibition effect associated with dependence of light power is further explained later in this disclosure with reference to FIGS. 4A to 4D.

After the injection of KOH etchant and potassium chloride (KCl) electrolyte, the etching time required for perforation (i.e., the irradiation time) depends on the remaining thickness H achieved during the pre-etching process. For example, a 120-s irradiation time is required for 114 nm of the remaining silicon thickness. Perforation leads to an over-etching stage characterised by rapid ramping in the current signal and an over-etching time that can be used to control the final pore size of nanopore structure 101. For instance, when using a silicon sample with a 19-nm remaining thickness, a 4.4-nm nanopore structure 101 can be fabricated using an irradiation time of 20 s and an over-etching time of 0.6 s with reference to FIGS. 1E to 1G.

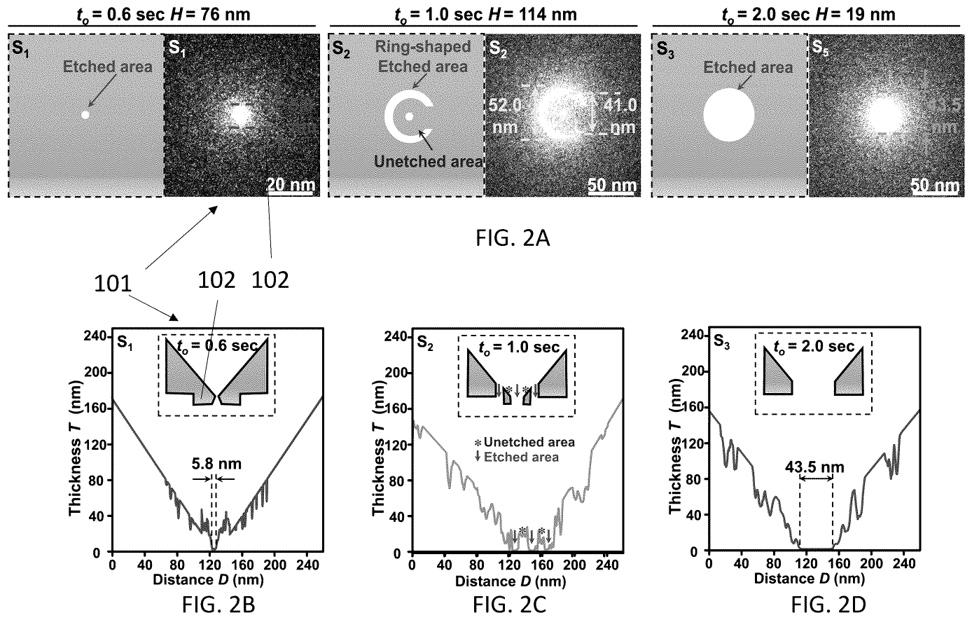

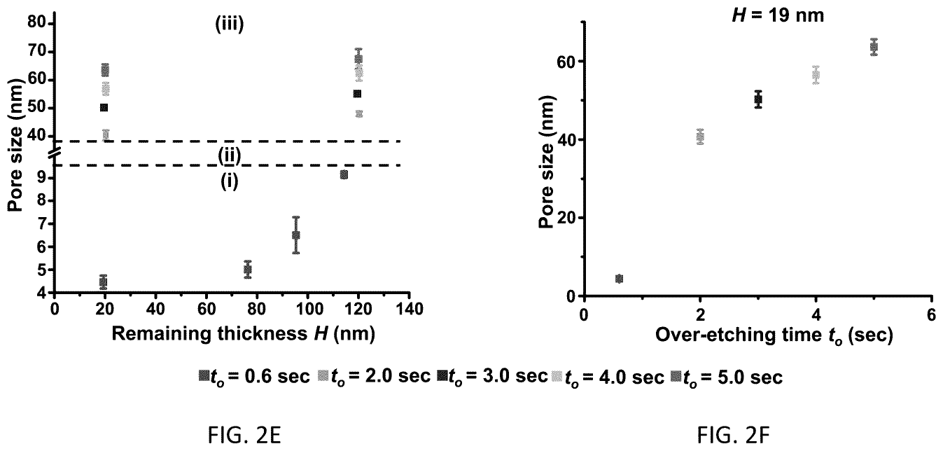

In an alternative example, the fabrication method may further comprise the step of performing over-etching and controlling an over-etching time after initial perforation of the aperture 102 to fine-tune a final diameter of the nanopore aperture. The overall structure resulting from the fabrication process, including its dimensions, can be visualized using techniques like TEM, as indicated generally in FIGS. 2A to 2F.

In an experiment conducted by the inventors, to validate the fabrication method, nanopore structures 100 with pore sizes ranging from 4.4 to 64.2 nm were fabricated using different combinations of remaining thicknesses (or irradiation times) and over-etching times. For instance, sub-10-nm pore sizes can be achieved using the same over-etching time of 0.6 s for different remaining thicknesses of silicon samples, such as 76 nm in S₁. To verify the photoinhibition effect, with reference to FIG. 2A, an overetching time of 1 s was tested on the remaining silicon thickness of 114 nm in S₂, resulting in a nanopore with a central perforation and ring-shaped etched area. Noteworthily, the etched pattern in S₂ was consistent with the calculated near-field diffraction pattern, confirming that the photoinhibition effect regulated the etching rate and area. Additionally, using an over-etching time longer than 1 s on a silicon sample with a 19-nm remaining thickness could aid the fabricate of nanopore structure with a greater than 40 nm pore size. The shapes and pore sizes of the example nanopore structures S₁, S₂, and S₃ were further verified using electron transmission intensity profiles as shown in FIGS. 2B, 2C, and 2D.

A correlation study was conducted to examine the relationship among the pore size, remaining thickness and over-etching time. Results reveal that the fabrication method offers three distinct working regions mainly governed by the overetching time. These regions include a sub-10 nm nanopore region using an overetching time of ≤0.6 s, an over-40-nm nanopore region using an over-etching time of ≥2 s and a transition region using an over-etching time between 0.6 and 2 s (referring to FIG. 2E). Because the silicon sample has a defined remaining thickness, the final pore size can be precisely controlled by fine-tuning the over-etching time as shown in FIG. 2F.

Additionally, a repeatability study assessing the MPSN fabrication method demonstrated that the measured pore sizes with an average standard deviation of about 1.1 nm could be achieved by using four identical fabrication conditions, i.e., the same over-etching time of 0.6 s utilised to etch three samples of the same remaining thickness with reference to FIGS. 2E and 2F.

The nanopore structure, also referred as a micro-pyramidal silicon nanopore (MPSN), characterized by its defined aperture size being less than or equal to 10 nm, more preferably less than 5 nm, with elongated dwell zone as earlier described in accordance with embodiments of the present invention, may be particularly suited for applications requiring enhanced sensitivity and resolution in single-molecule analysis.

Advantageously MPSN may be used for detection of molecular translocation events via current blockade signals. Alternatively, the MPSN may be employed to facilitate analysis under different translocation mechanisms and enables applications such as DNA or RNA sequencing, protein analysis (including antibody-antigen interactions, nanoparticle characterization (like the Au@PEG aggregates, and other biosensing tasks where increased interaction time (dwell time) is beneficial.

In one exemplary embodiment, to achieve controllable translocation of molecules, an MPSN device may be implemented to implement inertial kinetically regulated molecular translocation (i.e., inertial-kinetic translocation) in a laboratory-grade centrifuge. In this example, the MPSN device comprises the nanopore structure 101, and other components such as a centrifuge rotor, a centrifuge tube, an upper flow cell, a lower flow cell and Ag/AgCl electrodes. In this example, targeted molecules in the analyte flow from is translocated from the upper cell to the lower cell when subjected to the centrifugal forces and translocation of each of the modules will be sampled in form of an electrical signal by the Ag/AgCl electrodes at both the upper and lower flow cells.

The inventors devised that while the conventional electrokinetic translocation behaviour of molecules is governed by electrophoretic and electroosmotic forces in nanopores, the inertial-kinetic translocation of molecules is mainly regulated by centrifugal force, with superposed counter-balancing forces caused by electrokinetic effects in the MPSNs. This balanced state can be achieved by adjusting the pH value of the analyte medium and measuring the characteristic pH value for each molecule under test before the sensing experiment.

In this embodiment, when an MPSN is centrifuged, its pyramid design offers a favourable funnel-shaped centrifugal force field that effectively captures and guides molecules through the nanopore by overcoming Brownian motion and a potential barrier ΔU caused by molecule-pore interactions. Here, both the nanopore baseline current level I_{base} and the nanopore root-mean-square current noise I_{RMS} were proven to be independent of speed. Additionally, the deep pyramidal structure of the MPSNs (approximately 21.25-μm deep) offers an elongated sensing length supporting longer dwell times. The calculation indicates an approximately 115-nm sensing length for an MPSN with a 15-nm pore size.

The inventors conducted experiment to evaluate the advantages of the nanopore structure including the funnel-shaped entry in accordance with a preferred embodiment of the present invention.

For electrokinetic translocation in a solid-state nanopore system, the molecular capture radius can be described as:

$${R}^{′} = \frac{Q U}{k T} \frac{{D}_{p}}{8 + 2 π}$$

where Q is the surface charge of the trapped molecule), U is the applied bias (i.e., 300 mV), k is the Boltzmann constant, T is the environmental temperature, Dₚ is the pore size of the nanopore (i.e., 15 nm). Therefore, the capture radius of the nanopore to BSA is around 210 nm at pH=7.

For the inertial-kinetic translocation in the MPSN, the molecular behaviour is influenced by the interplay between centrifugal force and Brownian diffusion. This competition determines the capture radius R′ by comparing the relative magnitudes of molecular inertial potential energy and thermal energy. On the other hand, for the electrokinetic translocation in MPSN, the molecular capture radius R′ can be determined by comparing the relative magnitudes of molecular electrophoretic force and local friction drag, and therefore the capture radius can be described as:

$${R}^{′} = \frac{k T}{Q E ( {R}^{′} )}$$

To compare the capture radius (R′) of two translocation methods in MPSN, a molecular motion model was developed using COMSOL Multiphysics. The model utilized the Langevin equation to study molecular traces. The simulation area ranged from x=−25 μm to 25 μm and z=0 μm to 60 μm. A simulated protein with a molecular weight of 50 kDa, a diameter of 5 nm, and a surface charge was released within the simulated area. The simulation concluded when the protein exited the simulation area through translocation.

In the inertial-kinetic translocation simulation, the centrifugal force f_{c} is assumed to be constant throughout the chamber (f_{c}/f_{cmax}=1). The results demonstrate that the molecular translocation process is predominantly governed by inertial force, ensuring the proteins released within the pyramid structure with 20-μm thickness will be translocated through the nanopore. Thus, the capture radius R′ of the inertial-kinetic translocation in the MPSN is around 20 μm.

As for the electrokinetic translocation, the electrophoretic force f_{EP} is calculated through simulating the distribution of electric field intensity E in MPSN. The results show that the molecular capture possibility is 100% when the distance between the starting location of molecule and the nanopore centre is within the capture radius, i.e., 350 nm, where the molecular behavior is dominated by the electrokinetic force. Compared with the electrokinetic translocation, the inertial-kinetic translocation provides a longer capture radius in MPSN.

The measured current blockade signals clearly delineate three distinct stages related to the inertial-kinetic translocation in MPSNs: (i) molecule outside the sensing zone, (ii) molecule inside the sensing zone and (iii) molecule passing through the nanopore. The molecular motions in stages (i) and (ii) are governed by centrifugal force and Brownian motion, whereas centrifugal force and the potential barrier become dominant in stage (iii).

To achieve inertial-kinetic translocation of molecules, the nanopore needs to provide enough mechanical strength to withstand high levels of inertial force in centrifuge. In addition, a funnel-shaped nanopore is preferred to enable targets distributed over a large range for better capture and sense of molecules in nanopore. It was observed that a solid-state funnel-shaped nanopore of over tens micrometres thickness can meet these requirements, and such a nanopore can be fabricated by using glass pulling and silicon chemical etching techniques in accordance with embodiments of the present invention.

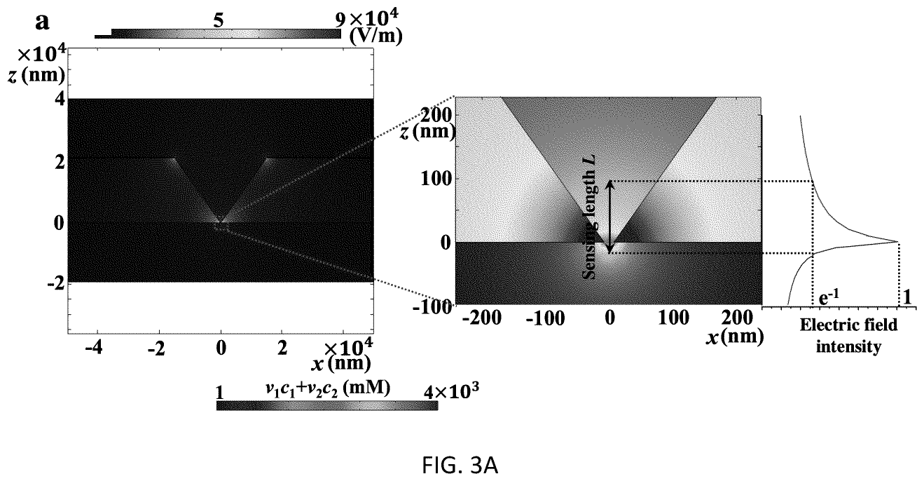

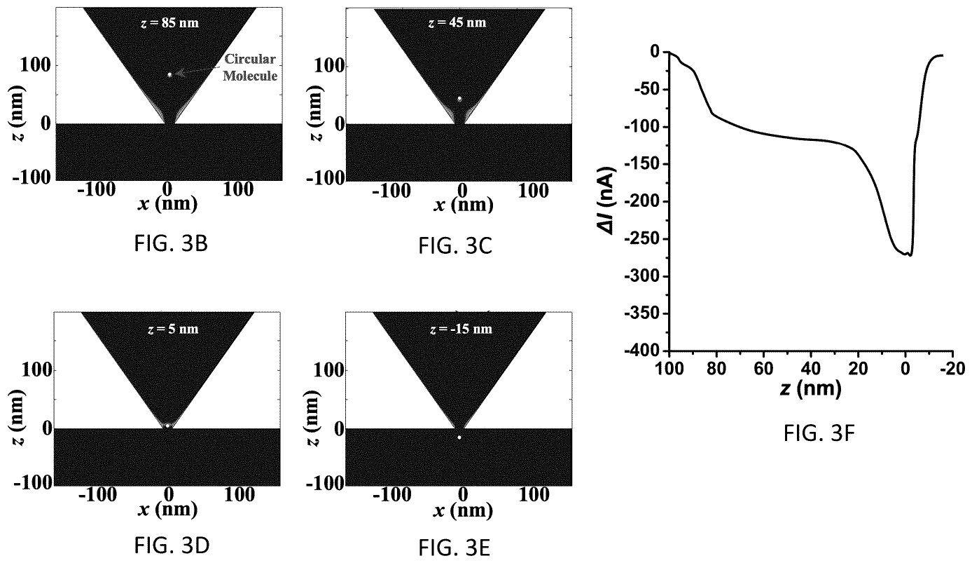

With reference to FIG. 3A, by implementing an ionic mass transport model in COMSOL, simulations were conducted to study the ion concentration change during the translocation of a circular molecule and therefore deduce the sensing area and length of MPSN. The distribution of electrical field and the net ionic concentration were calculated using the Helmholtz Equation and Nernst-Planck-Poisson (NPP) equation, respectively. In the simulation, the electrolyte was 1M KCl, the nanopore material was single crystal silicon, and the pore size was set at 15 nm. The current signals were calculated using the conductivity equation of the electrolyte⁴⁹, which can be described as:

I=∫_{S}F(v₁\vec{N₁}+v₂\vec{N₂})·\vec{n}dS

where F is the Faraday constant; v₃ and v_{%} represent the valences of potassium and chloride ions, respectively, while \vec{N₁} and \vec{N₂} represent the ionic flux of potassium and chloride ions, respectively; S is surface of the sensing area of the MPSN; \vec{n} is outward unit normal vector.

With reference also to FIGS. 3B to 3F, by simulating the translocation of a 7-nm circular molecule with a surface charge of 17e through a nanopore, the study reveals that the current drop begins when the molecule's centre is positioned at z=100 nm, and ends at −15 nm.

As described earlier in this disclosure, the photoinhibition-assisted KOH etching technique may also be referred as photovoltaic electrochemical etch-stop technique and used for micro and nano manufacturing. The mechanism of this technique is that the photovoltage induced by the separation of the photogenerated hole-electron pairs partially offsets the applied potential, resulting in a slower etching rate on light illuminated areas (bright areas) than these on dark areas at the silicon-KOH interface, as illustrated in FIG. 1A.

To illustrate the dependence of light power in this technique, a control experiment has been performed to study the silicon etching rate of KOH at room temperature using 460-nm narrowband light of different light power ranging from 0 to 0.9 W. The light is collimated using a convex lens to achieve a collimated beam size of 8.1 mm on the silicon samples, with reference to FIGS. 4A and 4B.

With reference to FIG. 4C, the microscope photographs of etched silicon samples using different etching time and light power is illustrated. From these photographs, we can further calculate the remaining silicon thickness H by measuring the square side length a_{etch} in the silicon pyramid.

Referring to FIG. 4D, the dependence of light power in the photoinhibition-assisted KOH etching technique, demonstrating that the etch rate at room temperature decreases with the increase of light power.

With reference to FIG. 5A, the micro-pyramid structure was simulated by COMSOL Multiphysics. For the pre-etching process, the simulation results were displayed in the form of optical distribution on a two-dimensional cross section through the pyramid tip. As shown in the plot in FIG. 5B, the simulation reflects that peak position of transmitted light blue shifts with the reduce of the thickness H of the remaining layer while light enters the model from the boundary Z₂. The distribution of <S> in the structure was calculated while monochromatic light incident as illustrated in FIG. 5A, and then incident wavelength was parametric scanned from 430 nm to 515 nm for simulating the wideband incident light with 120 nm bandwidth, enabling plot corresponding wideband transmission spectrum by space integrating <S> of different wavelength along Z₁.

With reference to FIGS. 5C and 5D, for the near-field etching process, the simulation reveals that the near-field (T<λ/2) transmission pattern changes with the further decrease of the thickness H from 200 nm to 10 nm while light with λ_{P}=475 nm enters the model from the boundary Z₁. The spatial distribution shown in FIG. 5C was obtained, whose near-field transmission pattern on the boundary Z₂ shows characteristic features of focusing on around 130 nm central area and increasing with reduce of H as shown in FIG. 5D. A bimodal diffraction distribution was displayed in the center of dozens of nanometers, and spatial distance between the two focusing areas trends to be decreased with the decrease of H. It is noted that the two-peaks distance is around 45 nm while H is around 100 nm, which has been confirmed by the diameter of ring-shaped unetched area in FIG. 2.

The equipment and materials used in experiments conducted by the inventors may be summarized as follows.

### The Preparation Process in MPSN Fabrication

Initially, two 200-nm-thick Si₃N₄ layers were deposited on both sides of a silicon sample using plasma-enhanced chemical vapour deposition (Oxford Instruments plc). Subsequently, two square patterns were transferred to the photoresist layers on both sides of the silicon through photolithography (SUSS MicroTec SE), which included standard procedures such as soft baking, UV exposure, developing and hard baking. The square pattern on the front side had a side length of 30 μm, whereas that on the backside had a side length of 800 μm. Carbon tetrafluoride (flow rate: 100 sccm; gas generation time: 20 min) and argon (flow rate: 5 sccm; gas generation time: 20 min) were then introduced into a plasma chamber (Plasma Etch Inc.) at a vacuum pressure of 100 mTorr to eliminate the exposed Si₃N₄ into a plasma environment excited by a radiofrequency electrostatic field (frequency: 3.0 MHz; output power: 29.6 W; radiofrequency generation time: 5 min). Finally, an inverted micro-pyramid structure was fabricated on the front side of the silicon sample by etching it with 30% (w/w) KOH in water heated to 80.0° C. on a hotplate.

### Optical System—Spectral Detection System and Electro-Optical System in the Etching Process

With reference to FIG. 6A, in the spectral detection system 600, 460 nm broadband blue light 602 (output power: 0.77 W) with a full-width-at-half-maximum (FWHM) bandwidth of 60 nm is incident from back side and collimated with a convex collimated lens 604 (collimated beam size: 8.1 mm). The light passes through an alignment glass 606 and an optical beam expander 612. A CMOS 614 and a spectrometer 616 (USB4000, Ocean Optics) with 0.1 nm spectral resolution was located on the front side to monitor the peak position change of transmission spectrum. The wafer, or Si sample 610, was etched from back side by 30% (w/w) KOH 608 at room temperature (~22.0° C.), which can real-time measure the etching process by monitoring blue shift of spectral peak λ to long-term monitor the etching process and precisely control the remaining thickness H. Here, the dependence of spectral peak λ position on remaining thickness H was aligned by a contact steeper.

With reference to FIG. 6B, in the electro-optical system 650, 460 nm narrowband blue light 618 (output power: 0.77 W) is incident from front side and collimated with a convex collimated lens 620 (collimated beam size: 8.1 mm), passing through the alignment glass 606, an electrolyte of 0.1M KCl 622, a platinum electrode 624, the Si sample 610, and the 30% w/w KOH 608, and near-field transmission fringe is distributed at the remaining silicon layer. The transmitted light is filtered by a short-pass filter 626 before reaching a GaAsP PD with zero-bias transimpedance amplifier 628. Reverse photovoltage VO was generated in the spot because of the directed motion of photogenerated holes and electrons by applying 800 mV forward bias Vp, enabling to regulate the etching rate and area and eventually the pore size of MPSN.

Preferably, the quartz glass with alignment pattern, functioning as the alignment glass 606, may be fixed in the spectral detection system 600 and electro-optical system 650 as a reference coordinate system for aligning the position of etched Si sample 610, in which the pattern is four chrome arrows with their tips spaced 30 μm apart, enabling align the top corners of the bottom of the pyramid structure etched in samples. The alignment accuracy of the methods was measured as 2 μm.

### Molecule Sample Preparation

The proteins used, including EpCAM, bovine serum albumin, streptavidin, EpCAM-antibody and amylase, were obtained from Thermo Fisher Scientific, Inc. Fragment antigen-binding was procured from Rockland Immunochemicals, Inc. The 4.8-nm spherical Au@PEGs were synthesised using a kinetically controlled seeded growth method. Initially, 3.5-nm-sized Au seeds were synthesised by injecting tetrachloroauric acid into a mixed solution of sodium citrate and tannic acid at 70° C. Subsequently, the size of the Au seeds was increased to 4.8 nm by diluting the seed solution and injecting aliquots of gold precursor.

The molecule samples, comprising proteins and gold nanoparticles, were initially dispersed in 1 M KCl. The pH values of the samples were then adjusted by injecting HCl and precisely calibrated using a pH metre (PH-3C, Yk Scientific Instrument Co., Ltd) with a ±0.01 pH resolution. The pH dependence of the Zeta potential of the tested samples was measured using a light scattering analyser (DelsaMax PRO, Beckman Coulter, Inc.). To determine the characteristic pH value of the balanced state between the electrophoretic and electroosmotic forces, nanopore sensing of the molecules at different pH values in MPSNs was performed by applying voltage only without the use of centrifugal forces. The characteristic pH value was identified as the corresponding value when the current blockade signal indicated minimal or no translocation events. This value was measured for each sample.

### COMSOL Simulation

The near-field diffraction pattern on the silicon/etchant interface was simulated using a two-dimensional axisymmetric model in COMSOL Multiphysics. Ring-shaped diffraction patterns with a dark centre area around the pyramid tip were observed for different remaining silicon thicknesses ranging from 10 nm to 200 nm. The diameter of the dark centre area was found to decrease with decreasing remaining thickness. The inertial-kinetic translocation process in MPSNs was simulated using COMSOL Multiphysics to understand the sensing region of the micro-pyramidal structure. In the COMSOL model, ion diffusion in the electrolyte was calculated using the Nernst-Planck equation in the transport of diluted species module. The distribution of the electric field in the nanopore structure was calculated using the Poisson equation in an electro-statics module by setting the applied voltages U to 0.3 V or 0.6 V, pore size to 15 nm and conductivity of 1 M KCl solution to 111,000 μS/cm. The sensing length of the nanopore was defined as the length between two points at which the maximal electric field intensity decays to its e⁻¹ value. Additionally, the distribution of funnel-shaped centrifugal force field and drag force generated on the molecules in the micro-pyramidal structure were calculated using Navier-Stokes equation in the laminar flow module.

### Modelling of Molecular Motions in MPSNs

During the molecular capturing process, molecular motions are governed by a competition between centrifugal force and Brownian diffusion and can thus be described using the Langevin equation. Moreover, based on the Einstein-Smoluchowski equation, the molecular diffusion coefficient is closely related to the molecular shape K′. Therefore, the capture duration t₁ can be calculated using the equation 6πμRK′L/f_{c}, where μ represents dynamic viscosity coefficient of molecules, R represents the equatorial semi-axis of molecules, L represents the sensing length and f_{c} represents the centrifugal force exerted on molecules. As for molecular motions during the translocation through nanopore process, translocation time t₂ (time until desorption) is a stochastic variable dependent on the bulk dissociation rate.

Thus, t₂ can be calculated using an Eyring-like form

$${t}_{0} {e}^{- \frac{h ( {f}_{c} )}{k T}} ,$$

where h represents the force-dependent factor, t₀ represents the mean of the exponential distribution, k represents Boltzmann constant and T represents the environmental temperature. Consequently, the capture duration t₁ and translocation time t₂ can be calculated using the following equations:

$${t}_{1} = \frac{6 π μ R ξ L}{ρ {ω}^{2}}$$

$${t}_{{2}^{=}} {t}_{0} {e}^{- \frac{h ( m ρ {ω}^{2} )}{k T}}$$

Here ξ is the shape-to-mass weighting factors. The fitting results lead to a sensing length L in MPSN of about 92 nm, which is close to the calculated value of 115 nm, and a force-dependent factor h of about 8.45 μm

These embodiments may be advantageous in that, an sensing device that incorporates an inertial force kinetically regulated molecular translocation method into a micro-pyramidal silicon nanopore (MPSN) to achieve precise fabrication of sub-5 nm silicon nanopores and controlled single-molecule translocation is provided. The MPSN may be fabricated using photovoltaic electrochemical etch-stop technique that leverages the local photo-voltage and its counter-etching effect (i.e., photoinhibition effect) induced by near-field optical irradiation and implements a high-precision electro-optical monitoring process.

Advantageously, the wet-etching technique enables a controllable, self-limited, and closed-loop chemical etching process capable of reliably and cost-effectively fabricating silicon nanopores with resolutions ranging from 65 nm to 4 nm. The resulting nanopores may have a thickness of 21.25 μm, providing excellent structural strength to withstand high inertial force.

The real-time discrimination of the mass and shape of protein molecules as well as longitudinal monitoring of molecular reactions and conformational changes by quantifying the dwell times and detecting characteristic features in current blockade traces were validated. The reliable fabrication of sub-5-nm silicon nanopores combined with the enhanced controllability suggests promising potential for accurate and efficient molecule fingerprinting.

In addition, the throughput of the MPSN device may be further improved through parallel fabrication and by monitoring 2D nanopore arrays. For example, in one example embodiment, the silicon nitride etch mask may be patterned with an array of squared openings that allows an etching of initial inverted micro-pyramid recesses using KOH, and thereby allowing an array of nanopore structure being fabricated after completing the etching process on the back-side of the (100) silicon substrate. The same photovoltaic electrochemical etch-stop technique may be applied to precisely control the etching process to form the necessary aperture and dwell zone in each of the nanopore structures formed on the silicon substrate. It should also be understood that the use of etching mask of other material being patterned using other lithography process may be employed in alternative embodiments, e.g. imprintable polymer-based etch mask may be use to replace silicon nitride hard mask if necessary, e.g. in a low-cost fabrication process.

Additionally, an intelligent data-driven close-loop mechanism may be used to increase the controllability and sensing capability of the in-tube MPSN device. This will enable automated and quantitative recognition of molecules and facilitate the spatiotemporally resolved sensing of molecule interactions.

One or more of the components and functions illustrated the figures may be rearranged and/or combined into a single component or embodied in several components without departing from the scope of the invention. Additional elements or components may also be added without departing from the scope of the invention.

It is to be understood that, if any prior art information is referred to herein, such reference does not constitute an admission that the information forms a part of the common general knowledge in the art.

1. A nanopore structure comprising:
an aperture extending through a substrate, wherein the aperture defines a central axis; and
a dwell zone defining the aperture, the dwell zone configured to increase a translocation time of an analyte passing through the aperture along the central axis.

2. The nanopore structure of claim 1, wherein the dwell zone corresponds to an elongated sensing length within the aperture and along the central axis.

3. The nanopore structure of claim 2, wherein the elongated sensing length is greater than a narrowest opening diameter of the aperture.

4. The nanopore structure of claim 3, wherein the aperture comprises a narrowest opening diameter of less than or equal to 4.4 nm.

5. The nanopore structure of claim 3, wherein the elongated sensing length is in a range of 90-115 nm.

6. The nanopore structure of claim 2, wherein the dwell zone is defined by a cylindrical tubular structure including the aperture extending therethrough along the central axis.

7. The nanopore structure of claim 1, further comprising an entry zone arranged to guide the analyte towards an entrance of the dwell zone with the narrowest opening diameter of the aperture.

8. The nanopore structure of claim 7, wherein the entry zone includes a funnel-shaped entry.

9. The nanopore structure of claim 8, wherein the entry zone includes a micro-pyramidal shape formed by anisotropic etching of the substrate.

10. The nanopore structure of claim 9, wherein the substrate includes a (100) silicon substrate.

11. A method for fabricating a nanopore structure, comprising:
forming an initial structure on a front-side of a (100) silicon substrate, wherein the initial structure includes a recess having a micro-pyramid shape; and
etching a portion of substrate material from a back-side of the (100) silicon substrate to form the nanopore structure including an aperture extending through the (100) silicon substrate at a tip of the recess on the front-side.

12. The method of claim 11, wherein the nanopore structure further includes a dwell zone being form by etching the back-side of the (100) silicon substrate, wherein the aperture is extended along an elongated sensing length defined by the dwell zone, and wherein the dwell zone is configured to increase a translocation time of an analyte passing through the aperture.

13. The method of claim 12, wherein the step of etching a portion of substrate material from the back-side of the (100) silicon substrate comprises the step of performing a photovoltaic electrochemical etch-stop process to regulate an etching rate of the substrate material at or proximate to the aperture of the nanopore structure.

14. The method of claim 13, wherein the photovoltaic electrochemical etch-stop process includes, under an electrical bias Vp applied across the front-side and the back side of the (100) silicon substrate, an application of a near-field optical irradiation at or proximate to the aperture of the nanopore structure to induce a photoinhibition effect so as to locally modulate the etching rate at or proximate to the aperture of the nanopore structure.

15. The method of claim 13, wherein the near-field optical irradiation includes narrowband blue light.

16. The method of claim 13, wherein the near-field optical irradiation is applied to the front-side of the (100) silicon substrate covering at least the tip of the recess to generation a near-field diffraction pattern around the tip, wherein a bright area is defined at or proximate to the tip.

17. The method of claim 16, wherein the near-field optical irradiation is applied to generate a photovoltage Vo induced by a separation of photogenerated electron-hole pairs which at least partially offsets the electrical bias Vp being applied to suppress the etching rate at the bright area.

18. The method of claim 11, further comprising the step of thinning the (100) silicon substrate to a predetermined remaining thickness prior to the step of etching a portion of substrate material from the back-side of the (100) silicon substrate to form the nanopore structure.

19. The method of claim 18, wherein remaining thickness is detected by monitoring transmission spectral characteristics including a shift in transmission spectral peaks of broadband blue light detected by a spectrometer at the front-side of the (100) silicon substrate, wherein the broadband blue light is irradiated on the back-side of the (100) silicon substrate during the step of thinning the (100) silicon.

20. The method of claim 18, wherein the (100) silicon substrate is etched by anisotropic wet etching using KOH.
